(19) United States
(12) Patent Application Publication
(10) Pub. No.: US 2021/0005292 A1
(43) Pub. Date: Jan. 7, 2021

(54) SYSTEM AND METHOD OF UTILIZING A USER'S HEALTH DATA STORED OVER A HEALTH CARE NETWORK, FOR DISEASE PREVENTION

(71) Applicant: Patientory, Inc., Atlanta, GA (US)

(72) Inventor: Chrissa Tanelia McFarlane, Atlanta, GA (US)

(21) Appl. No.: 16/583,143

(22) Filed: Sep. 25, 2019

Related U.S. Application Data
(60) Provisional application No. 62/736,376, filed on Sep. 25, 2018.

(51) Int. Cl. G16H 10/60; G06F 16/27; G16H 50/50; G06N 20/00

(52) U.S. Cl. CPC G16H 10/60; G06N 20/00; G16H 50/50; G06F 16/27

(57) ABSTRACT

A system and a method for utilizing a user's health data stored over a health care network, for disease prevention are described. The method comprises providing a user device for allowing the user to manage access of the user's health data to a third party. The user data is correlating, using a machine learning algorithm module, with similar data of other users for suggesting a next best action to be performed by the third party, for preventing occurrence of a disease to the user.

### OTHER RELATED APPLICATIONS

The present application is a U.S. Non-Provisional Patent Application claiming priority of U.S. Provisional Patent Application Ser. No. 62/736,376 filed on Sep. 25, 2018, which is hereby incorporated by reference.

### BACKGROUND OF THE INVENTION

### Field of the Invention

The present disclosure is generally related to access management and processing of health data stored over a blockchain network, and more particularly related to the processing of health data for disease prevention.

### Description of the Related Art

The subject matter discussed in the background section should not be assumed to be prior art merely as a result of its mention in the background section. Similarly, a problem mentioned in the background section or associated with the subject matter of the background section should not be assumed to have been previously recognized in the prior art. The subject matter in the background section merely represents different approaches, which in and of themselves may also correspond to implementations of the claimed technology.

To protect important information, utilizing storage on cloud networks is one approach to provide data redundancy. For sensitive information, the information may be stored in an encrypted form. Blockchain leverages both cloud networks and encryption to define storage of all information in a block wise manner. The blocks can be added to the blockchain in a linear and chronological order. Conventional techniques for sharing clinical data of a user with interested third parties consume significant time. Further, there does not exist, and therefore a need exists for, a common platform where the user could easily manage access of user data with interested third parties, particularly where different access rights could be assigned to each interested third party. Further, a method and a system utilizing whereby a user could charge for allowing access of his data is also desired.

Applicant believes that a related reference corresponds to U.S. patent No. 2019/0088373 for an Automated Assistant for Remote Patient Tracking, Diagnosing, Alerting and Prevention of Heart diseases, Cardio Warning Service/System. Applicant believes another related reference corresponds to U.S. Pat. No. 10,360,495 for an Augmented Reality and Blockchain Technology for Decision Augmentation Systems and Methods using Contextual Filtering and Personalized Program Generation. None of these references, however, teach of the usage of a blockchain to securely store patient data and share the stored patient data with desired third parties that may aid in preventing of diseases upon an analysis of the patient data. Further, the patient data is easily and readily accessible to permitted parties with different access rights as determined by the patient.

### SUMMARY OF THE INVENTION

It is one of the objects of the present invention to provide a system and method of utilizing a user's health data stored over a health care network for aiding in disease prevention.

It is another object of the present invention to provide a system a system and method of utilizing a user's health data stored over a health care network for aiding in disease prevention that stores the health data via a blockchain network.

It is still an object of the present invention to provide a system a system and method of utilizing a user's health data stored over a health care network for aiding in disease prevention that stores and encrypts the health data of the user.

It is still another of the present invention to provide a system a system and method of utilizing a user's health data stored over a health care network for aiding in disease prevention that allows retrieval of stored data remotely.

It is another object of the present invention to provide a system a system and method of utilizing a user's health data stored over a health care network for aiding in disease prevention that allows the user to manage access of their health data to appropriate parties.

It is yet another object of the present invention to provide a system a system and method of utilizing a user's health data stored over a health care network for aiding in disease prevention that uses machine learning to analyze the user's data and correlate similar health data of other users for suggesting a next best action to be performed by appropriate parties to prevent the occurrence of a disease to a user.

It is another object of the present invention to provide a system a system and method of utilizing a user's health data stored over a health care network for aiding in disease prevention that allows for easy monetary transactions.

It is still another object of the present invention to provide a system a system and method of utilizing a user's health data stored over a health care network for aiding in disease prevention that aids in prolonging the life of the user.

It is another object of the present invention to provide a system a system and method of utilizing a user's health data stored over a health care network for aiding in disease prevention that helps the users monitor their health over predetermined periods of time.

It is an object of the present invention to provide a system a system and method of utilizing a user's health data stored over a health care network for aiding in disease prevention that can easily be implemented into mobile devices for quick and easy data collection and accessing.

It is yet another object of the present invention to provide an invention that is inexpensive to implement and maintain while retain its effectiveness.

Further objects of the invention will be brought out in the following part of the specification, wherein detailed description is for the purpose of fully disclosing the invention without placing limitations thereon.

### BRIEF DESCRIPTION OF THE DRAWINGS

The accompanying drawings illustrate various embodiments of systems, methods, and embodiments of various other aspects of the disclosure. Any person with ordinary skills in the art will appreciate that the illustrated element boundaries (e.g. boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another, and vice versa. Furthermore, elements may not be drawn to scale. Non-limiting and non-exhaustive descriptions are provided with reference to the following drawings. The components in the figures are not necessarily to scale, emphasis instead being placed upon illustrating principles.

FIG. 1 illustrates a network connection diagram of a Health Information Exchange (HIE) system for utilizing a user's health data stored over a health care network, for disease prevention, according to various embodiments.

FIG. 2 illustrates a method for symmetric encryption of data, according to various embodiments.

FIG. 2A illustrates a method for asymmetric encryption of data, according to various embodiments.

FIG. 3 illustrates a method for hybrid encryption of data, according to various embodiments.

FIG. 4 illustrates a system for storing and accessing data in a clinical health care network, according to various embodiments.

FIG. 5 illustrates a system for storing and accessing data in a clinical health care network implemented specifically over a blockchain network, according to various embodiments.

FIG. 6 illustrates a flow chart showing a process performed by a user for managing access of the user's health data, according to various embodiments.

FIG. 7 illustrates a flow chart showing an example process for use of a clinical network base module, according to various embodiments.

FIG. 8 illustrates a flowchart showing an example process for use of a support module e.g., clinical network support module, according to various embodiments.

FIG. 9 illustrates a flowchart showing an example process for use of a clinical machine learning algorithm module, according to various embodiments.

FIG. 10 illustrate exemplary data of correlations performed by the clinical machine learning algorithm module.

FIG. 10A illustrate exemplary data of correlations performed by the clinical machine learning algorithm module.

FIG. 11 illustrates a flowchart showing an example process for use of a hospital network base module, according to various embodiments.

FIG. 12 illustrates exemplary data stored in the machine learning database, according to various embodiments.

FIG. 13 illustrates exemplary data stored in a support database, according to various embodiments.

FIG. 14 illustrates exemplary data stored in a clinical database, according to various embodiments.

### DETAILED DESCRIPTION OF THE EMBODIMENTS OF THE INVENTION

Some embodiments of this disclosure, illustrating all its features, will now be discussed in detail. The words “comprising,” “having,” “containing,” and “including,” and other forms thereof, are intended to be open ended in that an item or items following any one of these words is not meant to be an exhaustive listing of such item or items, or meant to be limited to only the listed item or items.

It should also be noted that as used herein and in the appended claims, the singular forms “a,” “an,” and “the” include plural references unless the context clearly dictates otherwise. Although any systems and methods similar or equivalent to those described herein can be used in the practice or testing of various embodiments of the present disclosure, various embodiments of the systems and methods will be described.

Embodiments of the present disclosure will be described more fully hereinafter with reference to the accompanying drawings in which like numerals may represent like elements throughout the several figures, and in which various example embodiments are shown. Various embodiments may, however, be embodied in many different forms and should not be construed as limited to the embodiments set forth herein. The examples set forth herein are non-limiting examples and are merely examples among other possible examples.

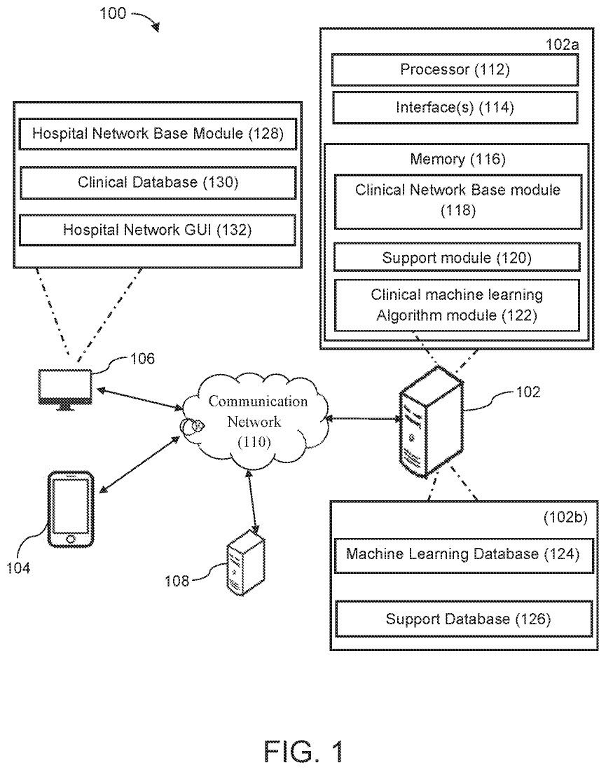

FIG. 1 illustrates a network connection diagram 100 of a Health Information Exchange (HIE) system 102 for utilizing a user's health data for disease prevention. The HIE system 102 may be connected with a user device 104, a service provider device 106, and a financial platform 108 (e.g., coin market), through a communication network 110.

The communication network 110 may be a wired and/or a wireless network. The communication network 110, if wireless, may be implemented using communication techniques such as Visible Light Communication (VLC), Worldwide Interoperability for Microwave Access (WiMAX), Long Term Evolution (LTE), Wireless Local Area Network (WLAN), Infrared (IR) communication, Public Switched Telephone Network (PSTN), Radio waves, and other communication techniques known in the art.

The HIE system 102 may comprise a group of components 102a required for utilizing the user's health data stored over a health care network, for disease prevention. The group of components 102a may include a processor 112, interface(s) 114, a memory 116, a clinical network base module 118, a support module e.g., a clinical network support module 120, and a clinical machine learning algorithm module 122.

The processor 112 may execute an algorithm stored in the memory 116 for utilizing the user's health data for disease prevention. The processor 112 may also be configured to decode and execute any instructions received from one or more other electronic devices or server(s). The processor 112 may include one or more general-purpose processors (e.g., microprocessors) and/or one or more special purpose processors (e.g., digital signal processors (DSPs), System On Chips (SOCs), Field Programmable Gate Arrays (FPGAs), or Application-Specific Integrated Circuits (ASICs)). The processor 112 may be configured to execute one or more computer-readable program instructions, such as program instructions to carry out any of the functions described in this description.

The interface(s) 114 may help an operator to interact with the HIE system 102. The interface(s) 114 may either accept inputs from users or provide outputs to the users or may perform both the actions. In various embodiments, a user can interact with the interface(s) 114 using one or more user-interactive objects and devices. The user-interactive objects and devices may comprise user input buttons, switches, knobs, levers, keys, trackballs, touchpads, cameras, microphones, motion sensors, heat sensors, inertial sensors, touch sensors, or any combination of the above. Further, the interface(s) 114 may be implemented as a Command Line Interface (CLI), a Graphical User Interface (GUI), patient GUI, a voice interface, or a web-based user-interface.

The memory 116 may include, but is not limited to, fixed (hard) drives, magnetic tape, floppy diskettes, optical disks, Compact Disc Read-Only Memories (CD-ROMs), and magneto-optical disks, semiconductor memories, such as ROMs, Random Access Memories (RAMs), Programmable Read-Only Memories (PROMs), Erasable PROMs (EPROMs), Electrically Erasable PROMs (EEPROMs), flash memory, magnetic or optical cards, or other type of media/machine-readable medium suitable for storing electronic instructions. The memory 116 may comprise modules implemented as a program.

In various embodiments, several users may interact with the HIE system 102, using the user device 104. Although a single user device has been illustrated, several user devices could similarly be connected to the communication network 110. Further, each of the user devices may have a device ID. In various embodiments, the device ID may be a unique identification code such as an International Mobile Equipment Identity (IMEI) code or a product serial number. It should be noted that a user may use a single user device or multiple user devices. Further, multiple users may use a single user device or multiple user devices. Further, the one or more users may receive and/or provide healthcare related products and services. The one or more users may include, for example and not limited to, patients, family and friends of the patients, hospitals, physicians, nurses, specialists, pharmacies, medical laboratories, testing centers, insurance companies, or Emergency Medical Technician (EMT) services.

The user device 104 may be a mobile, stationary device, a portable device, or a device accessed remotely. The visual graphical element such as, but not limited to, a barcode, text, a picture, or any other forms of graphical authentication indicia. In various embodiments, the barcode may be one-dimensional or two-dimensional. Further, the imaging device may include a hardware and/or software element. In various embodiments, the imaging device may be a hardware camera sensor that may be operably coupled to the user device 104. In various embodiments, the hardware camera sensor may be embedded in the user device 104. In various embodiments, the imaging device may be located external to the user device 104. In various embodiments, the imaging device may be connected to the user device 104 wirelessly or via a cable. It should be noted that image data of the visual graphical element may be transmitted to the user device 104 via the communication network 110.

In various embodiments, the imaging device may be controlled by applications and/or software(s) configured to scan a visual graphical code. In various embodiments, a camera may be configured to scan a QR code. Further, the applications and/or software(s) may be configured to activate the camera present in the user device 104 to scan the QR code. In various embodiments, the camera may be controlled by a processor natively embedded in the user device 104. In various embodiments, the imaging device may include a screen capturing software (for example, screenshot) that may be configured to capture and/or scan the QR code on a screen of the user device 104.

In various embodiments, a group of databases 102b may be connected to the HIE system 102. In various embodiments, the group of databases 102b may be implemented over a blockchain network (such as a PTOYNet blockchain network or a PTOYNet Ethereum™ Blockchain network) and may be present as different databases installed at different locations. The group of databases 102b may include a machine learning database 124 and a support database 126. The group of databases 102b may be configured to store data belonging to different users and data required for functioning of the HIE system 102. Different databases may be used in accordance with various embodiments; however, a single database may also be used for storing the data. Usage of the different databases may also allow segregated storage of different data and may thus reduce time to access desired data. In various embodiments, the data may be encrypted, time-dependent, piece-wise, and may be present as subsets of data belonging to each user. In various embodiments, the data may represent the results of one medical test in a series of multiple medical tests.

In various embodiments, the group of databases 102b may operate collectively or individually. Further, the group of databases 102b may store data as tables, objects, or other structures. Further, the group of databases 102b may be configured to store data required or processed by the HIE system 102. The data may include, but not limited to, a patient medical history, medical charts, medications, prescriptions, immunizations, test results, allergies, insurance provider, or billing information. Further, the data may be time-dependent and piece-wise. Further, the data may represent a subset of data for each patient. In various embodiments, the data may represent results of a medical test in a series of multiple medical tests. Further, the data may be securely stored. In various embodiments, the data may be encrypted.

In various embodiments, information stored in the group of databases 102b may be accessed based on users' identities and/or the users' authorities. The users' identities may be verified in one or more ways such as, but not limited to, biometric authentication (or bio-authentication), password or PIN information, user device registrations, a second-level authentication, or a third-level authentication. In various embodiments, the users' identities may be verified by the HIE system 102. Information provided by the users in a real-time may be used, by the HIE system 102, to confirm the users' identities. In various embodiments, the users' identities may be verified using a name, a password, one or security questions, or a combination thereof. In various embodiments, a user may be identified using an encryption key and/or a decryption key.

In various embodiments, the data stored in the group of databases 102b may be accessed at different levels, for example using a first level subsystem and a second level subsystem. In various embodiments, a user may directly access the first level subsystem. To access data stored in the second level subsystem, the second level subsystem may need to be accessed through the first level subsystem. It should be noted that the communication between the first level subsystem and the second level sub-system may be encrypted. In various embodiments, the second level subsystem may be implemented over a blockchain network (such as a PTOYNet blockchain network). In various embodiments, the PTOYNet blockchain network may be used to implement smart contracts.

In various embodiments, the service provider device 106 may comprise a hospital network base module 128, clinical database 130, and a hospital network Graphical User Interface GUI 132. The hospital network base module 128, clinical database 130, and the hospital network GUI 132 can be used for managing and accessing hospital network information.

In various embodiments, a primary care physician may input data into the HIE system 102 through the user device 104. The data may be processed by the first level subsystem and the second level subsystem. The data may be stored on the first level subsystem and/or the second level subsystem of the HIE system 102. The data may include, but not limited to, one or more instructions to a see patient's reports. Further, the data may be stored in one or more blockchains of the second level subsystem. The patient may be able to access the data relating to the patient's care provided by the primary care physician. The patient may be able to retrieve the data using the user device 104 of the patient.

In accordance with various embodiments, the patient may communicate with the providers using the HIE system 102. The providers may be individuals belonging to hospitals, or doctors and insurers. It should be noted that the providers may be able to access the data of the patient from the first level subsystem and/or the second level subsystem. Further, the physician specialist may be able to communicate with the patient. It should be noted that some, all (or substantially all) communications between patients and providers may be stored and may be accessible on a blockchain network.

In an embodiment, the patient may be the user that may receive healthcare related products or services and their personal data may be stored and used by hospitals, physicians, nurses, specialists, pharmacies, medical laboratories and testing centers, insurance companies, EMT services, etc. The user device 104 may be a remote device, a computer, a laptop, a tablet, a mobile phone, a smartphone, a smart watch etc. The Graphical User Interface (GUI) present on the user device 104 may allow the user to input the various user options when creating an account as well as allow or deny access to their patient data.

In various embodiments, the clinical network base module 118 may store Electronic Health Record (EHR) data. The user may be a patient. A user may create an account using a user device 104. The user may use personal identification information, such as a telephone number and/or an email address, to create an account. The clinical network base module 118 may store any personal information in one or more databases. After receiving the personal information from the user, the clinical network base module 118 may create a digital wallet such as a PTOYNet wallet and private key for the user.

In various embodiments, the clinical network base module 118 may store the private key on user devices selected by the user. The private key may be for user identification and authorization. In various embodiments, the private key may only be used by the user. The clinical network base module 118 may store corresponding public keys on one or more databases. The core service component may communicate with third-party servers and databases; also, it may be in communication with the servers and databases of a hospital computing network. The Remote Procedure Call (RPC) may communicate signed transactions from the user device 104 to the blockchain Node (e.g., the Quorum blockchain node). The signed transaction may grant permission to a hospital representative to view the patient's data. The RPC can communicate the signed transaction to the blockchain Node (e.g., the Quorum blockchain node), once the blockchain Node (e.g., the Quorum blockchain node) activates one or more smart contracts after confirming ownership of the signed transaction. The blockchain Node (e.g., the Quorum blockchain node) may revise a state of one or more blockchains and the blockchain Node (e.g., the Quorum blockchain node) may contain a hash function for the patient blockchain.

In various embodiments, the clinical network support module 120 may collect the relevant patient data from a plurality of users by using the public keys from the users. The public keys may be passed to the individual user devices to receive the user's patient data that may be stored in the clinical machine learning database 124. The user's patient data may be used in the clinical machine learning algorithm module 122 to determine if there are any correlations between various data points. In various embodiments, the clinical machine learning algorithm module 122 may use numerous correlations engines to correlate data. If any correlated data points are found, the clinical network support module 120 may receive the correlated data points from the clinical machine learning algorithm module 122 and may store the correlated data points in the clinical network support database 126. Data from the clinical network support database 126 may be sent to the hospital network base module 128.

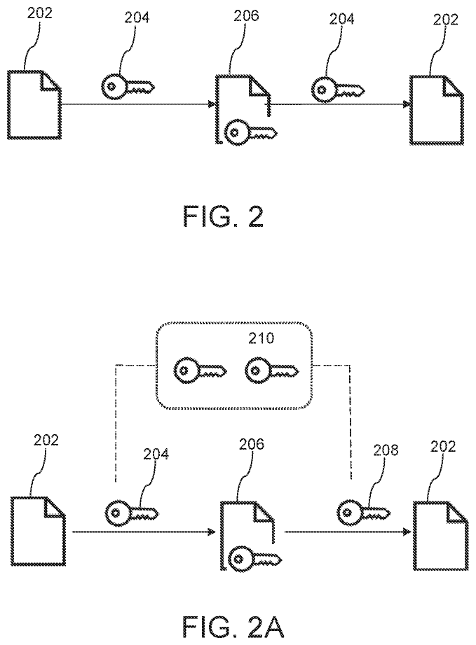

FIG. 2 illustrates a method for symmetric encryption of data, according to various embodiments. Original data 202 may be encrypted using a key 204 to obtain an encrypted data 206. The encrypted data 206 may be decrypted using the key 204 to obtain back the original data 202. It should be noted that encryption and decryption of the data may be performed using a same key. Further, one or more parties involved in a communication may have the same key to encrypt and decrypt the data.

FIG. 2A illustrates a method for asymmetric encryption of data, according to various embodiments. Original data 202 may be encrypted using a key 204 to obtain encrypted data 206. The encrypted data 206 may be decrypted using another key 208 to obtain the original data 202. It should be noted that encryption and decryption of the data may be performed using different keys e.g., a key pair 210.

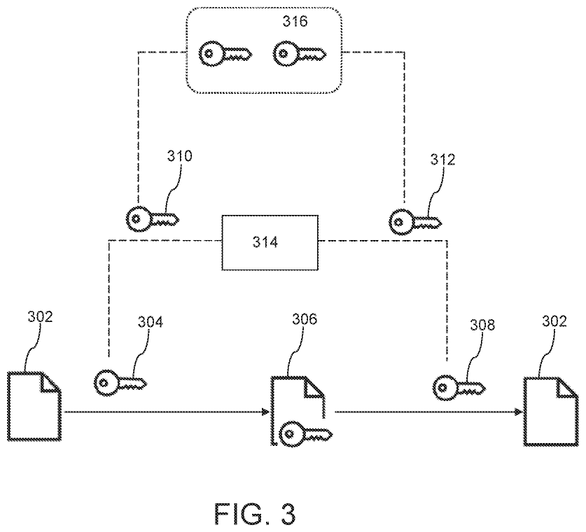

FIG. 3 illustrates a method for hybrid encryption of data, according to various embodiments. Both symmetric encryption and asymmetric encryption techniques may be used in tandem. In various embodiments, the symmetric encryption technique may be used to encrypt data 302 using a symmetric key 304 for producing encrypted data 306. The encrypted data 306 may be decrypted using another symmetric key 308 for obtaining data 302 or back data. Further, a public key 310 may be used to encrypt the symmetric key 304 and a private key 312 may be used to encrypt the symmetric key 308, stored as an encrypted key 314. The public key 310 and the private key 312 may form a key pair 316.

Further, the HIE system 102 may comprise the machine learning database 124, which may contain account information and activity of each user linked to the HIE system 102. In various embodiments, the account information and activity may include, for example, location, identifying information, and data relationships of the users with the providers. It should be noted that, the users added to the HIE system 102 may be in a direct relationship to the value of the system and hence value to the cryptocurrency.

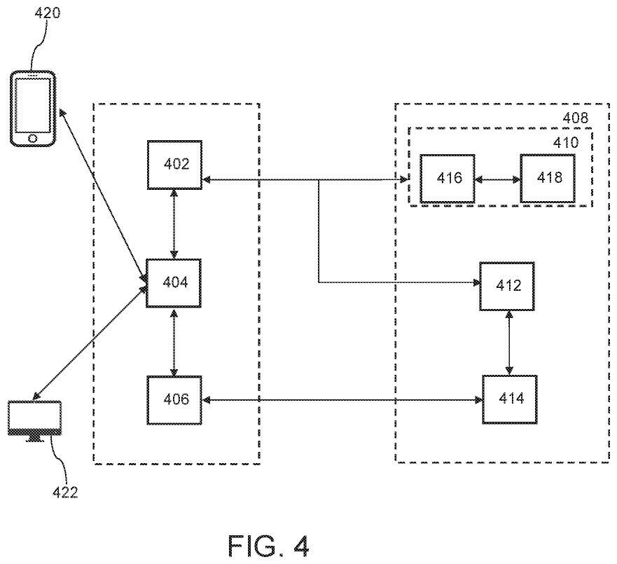

In accordance with various embodiments, FIG. 4 illustrates a system for storing and accessing data in a health care network, the first level subsystem may include a core service component 402 and a Remote Procedure Call (RPC) component 404. In various embodiments, the second level subsystem may include a blockchain node (e.g., the quorum blockchain node) component 406. In various embodiments, the first level subsystem may include the core service component 402, and the second level subsystem may include the RPC component 404 and the quorum blockchain node component 406. Further, the core service component 402 of the first level subsystem may be present in communication with third-party servers and databases of a hospital computing network 408. The hospital computing network 408 may include an Inter Planetary File System (IPFS) module 410, an EHR synchronization service 412, and a blockchain node (e.g., the quorum blockchain node) 414. Further, the IPFS module 410 may include an IPFS manager 416 and an IPFS node 418. The quorum blockchain node component 406 of the second level subsystem may communicate with the quorum blockchain node 414 of the hospital computing network 408. Patients may access the health care network for storing data through a user device 420, and a representative of a hospital may access the health care network through another user device 422.

In accordance with various embodiments, the representative of the hospital may want to synchronize Electronic Health Record (EHR) data of a patient. The first level subsystem and the second level subsystem may ask the patient for permission to allow a representative of the hospital to store the EHR data of the patient, through the IPFS module 410. Based at least on the permission granted by the patient, a signed transaction may be created to confirm the permission of the hospital to store the EHR data. Further, the signed transaction may activate a smart contract that may add hospital identification information such as a blockchain address to a list of permitted users.

In accordance with various embodiments, the signed transaction may be transmitted from the user device to the RPC component 404 of the first level subsystem and/or the second level subsystem. The RPC component 404 may communicate the signed transaction to the quorum blockchain node component 406 of the second level subsystem. The quorum blockchain node component 406 may activate one or more smart contracts. Thereafter, the quorum blockchain node component 406 may revise a state of one or more blockchains.

In accordance with various embodiments, based at least on the permission granted by the patient, the EHR synchronization service may obtain a list of patients from the RPC component 404. Further, the EHR synchronization service may confirm whether the patient has granted permission. Based at least on the permission, the first level subsystem and the second level subsystem may obtain the EHR data and may calculate a hash function for the EHR data. The HIE system 102 may match the hash function of the EHR data with a hash function for the patient blockchain on the quorum blockchain node component 406 of the second level subsystem. If the hash function of the EHR data matches with the hash function for the patient blockchain on the quorum blockchain node component 406 of the second level subsystem, the EHR data of the patient may remain unchanged.

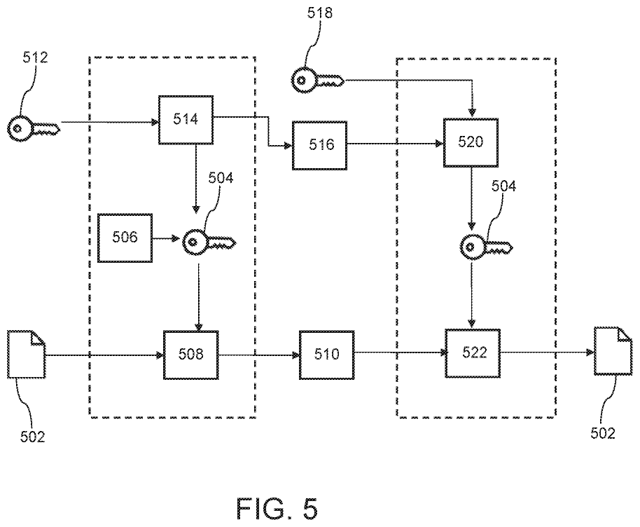

In accordance with various embodiments, FIG. 5 illustrates a system for storing and accessing data in a health care network implemented specifically over a blockchain network (such as a PTOYNet blockchain network or a PTOYNet Ethereum™ blockchain network), the HIE system 102 may execute an application for determining permission from the user for obtaining EHR data 502. In various embodiments, if the user grants the permission, the HIE system 102 may obtain the EHR data 502 for calculating a hash function for the EHR data 502. Further, the HIE system 102 may match the hash function of the EHR data 502 with a hash function for the user blockchain on the quorum blockchain node of the second level sub-system. In various embodiments, if the two hash matches, there is no change to the user's EHR data 502. In various embodiments, the two hash functions do not match, the HIE system 102 may generate a random string (e.g., secret key) 504, through a random key generator 506. The secret key 504 may be used for Advanced Encryption Standard (AES) encryption of the EHR data 502, in an AES encryptor 508, for generating encrypted EHR data 510.

In accordance with various embodiments, key 504 may then be encrypted, for example, by a Rivest-Shamir-Adleman (RSA) public key 512 of the patient, in an RSA encryptor 514, to generate an encrypted secret key 516. The HIE system 102 may also send the encrypted EHR data 510 to the core service component 402 for forwarding the data to the IPFS manager 416 of the hospital computing network 408 for storage. The IPFS manager 416 may send an IPFS hash function to the core service component 402 for further sending the IPFS hash function to EHR synchronization service 412. The EHR synchronization service 412 may further update the patient smart contract with the new IPFS hash function, the encrypted random key, a hash function of the unencrypted file, and file name.

In accordance with various embodiments, a hospital representative, such as a doctor or a hospital administration, may want to view the EHR data 502. In such a scenario, the user may first send a signed transaction to a RPC component 404 for granting permission to the hospital representative to view the EHR data 502. Once the permission is granted, the signed transaction may be added to the quorum blockchain node 414 and a new smart contract will be created for a blockchain corresponding to the hospital representative. After adding the signed transaction, the hospital representative may be able to view the EHR data 502 of the user, on a device.

In various embodiments, in order to view the EHR data 502 on the device, the HIE system 102 may collect the encrypted EHR data 510 from the user's blockchain and may decrypt the encrypted EHR data 510 using patient's RSA private key 518. The HIE system 102 may decrypt the encrypted secret key 516, in an RSA decryptor 520, using RSA private key of the hospital representative. The encrypted EHR data 510 may be decrypted using the RSA public key 512 of the hospital representative, in an AES decryptor 522. Further, the HIE system 102 may load the decrypted EHR data 502 to the smart contract previously created for the hospital representative.

After loading the decrypted EHR data 502, the RPC component 404 may obtain the signed transaction from the patient's user device and transmit the signed transaction to the quorum blockchain node component 406 of the second level subsystem. The quorum blockchain node component 406 may confirm ownership of the signed transaction and may execute the smart contract for the hospital representative to view the user's data.

In various embodiments, the patient may decline permission for the hospital representative to have access to the EHR data 502. For example, the user through a user device may send a signed transaction revoking permission to the RPC component 404. The RPC component 404 may forward the signed transaction to the quorum blockchain node component 406 of the second level subsystem. The quorum blockchain node component 406 may confirm ownership of the signed transaction and may delete the smart contract previously created to allow the hospital representative to have access to the patient's EHR data 502.

Further, the HIE system 102 may comprise a clinical health record network for an intermediary, enabling sharing of user's medical records with providers. The user may grant specific permissions to modify the user's medical records in the clinical database 130. In various embodiments the user may be individuals constituting a value chain, such as patients, doctors, nurses etc. In various embodiments, the user may be remote doctors logging into the HIE system 102 or doctors present in hospitals.

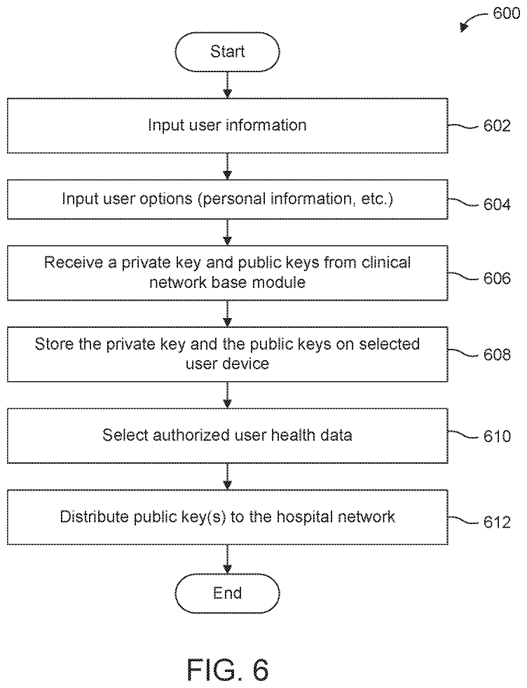

An example process performed by a user for managing access of the user's health data will now be explained with reference, for example, to the flowchart 600 shown in FIG. 6. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

Referring to FIG. 6, the user may send a request to create a user account through the clinical network base module 118, at step 602. The user may input the user information, such as personal information, e-mail, and address, through the GUI present on the user device 104, at step 604. The user may receive the private key and the public keys from the clinical network base module 118, at step 606. The private key may allow the user to access the user's health data. The public key may allow the user to provide access, to a third party, a portion or selected section of the user's health data while blocking the third party from accessing patient data that is unauthorized by the user. The third party may correspond to at least one of a doctor, surgeon, and a physician present in a hospital network.

Both the private key and the public keys may be stored on a selected user device, at step 608. The selected user device could be the user device 104 or a separate user device. The user may then select authorized user health data accessible by the third party and the public keys for accessing the authorized user health data, at step 610. The public keys may then be distributed to the third party, at step 612. Further, access of the authorized user health data may be provided to the third party.

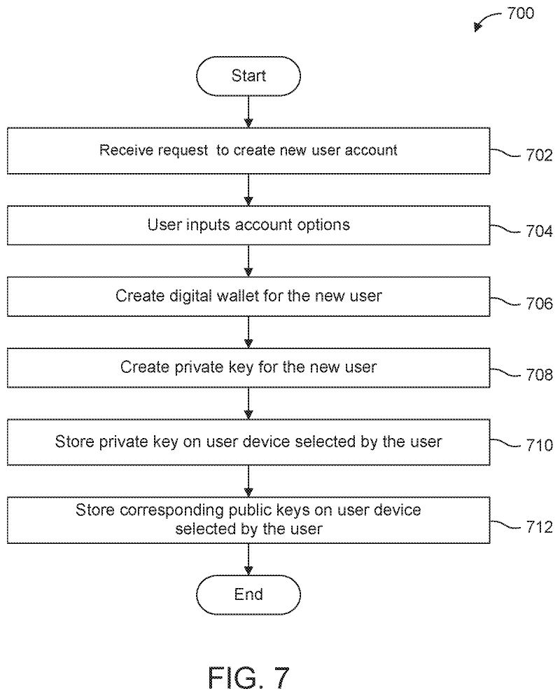

An example process utilizing the clinical network base module 118 will now be explained with reference to, for example, the flowchart 700 shown in FIG. 7. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

Referring to FIG. 7, a request may be received to create a new user account, at step 702. The request may be provided by the user. The user may input his account options via the GUI of the user device 104, at step 704. The account options may comprise personal information of the user, such as, for example but not limited to, e-mail address, address, name, etc. The clinical network base module 118 may create a digital wallet for the new user, e.g., a PTOYNet Wallet (e.g., a PTOYNet Ethereum Wallet), at step 706. The clinical network base module 118 may then create a private key specific for a new user, at step 708. The private key may provide access to the user's health data stored on the digital wallet. The private key may then be stored on selected user devices at step 710. This can provide the function of only allowing the user to access the private key and thus have access to user's health data. Corresponding public keys may be stored on the user devices which may be selected by the user, to allow the user to send a public key to the Hospital Network, to provide access to the user's health data, at step 712.

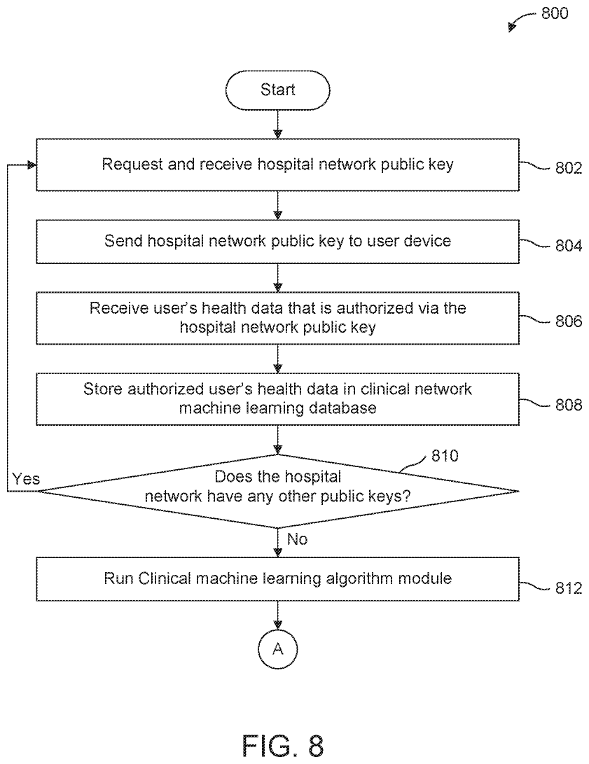

An example process utilizing the clinical network support module 120 will now be explained with reference to, for example, the flowchart 800 shown in FIG. 8. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

Referring to FIG. 8, the clinical network support module 120 may request and receive hospital network public keys at step 802. Such keys may correspond to the public keys that users may provide to the hospital network to grant access to a portion of the user's patient data. The clinical network support module 120 may then send the hospital network public key to the user device 104 of the user, at step 804. Alternatively, the hospital network public key may be sent to another user device that the user may have selected while creating the account. The clinical network support module 120 may receive the user's health data that is authorized and accessed by using the hospital network public key and the user using his private key, at step 806. The authorized user's health data may then be stored in the clinical machine learning database 124, at step 808.

The clinical network support module 120 may determine if the hospital network has any other public keys for additional user's health data, at step 810. If the hospital network does not have any remaining public keys for additional users, the clinical machine learning algorithm module 122 may be executed, at step 812. If the Hospital Network does have any additional public keys, the control may be transferred back to step 802.

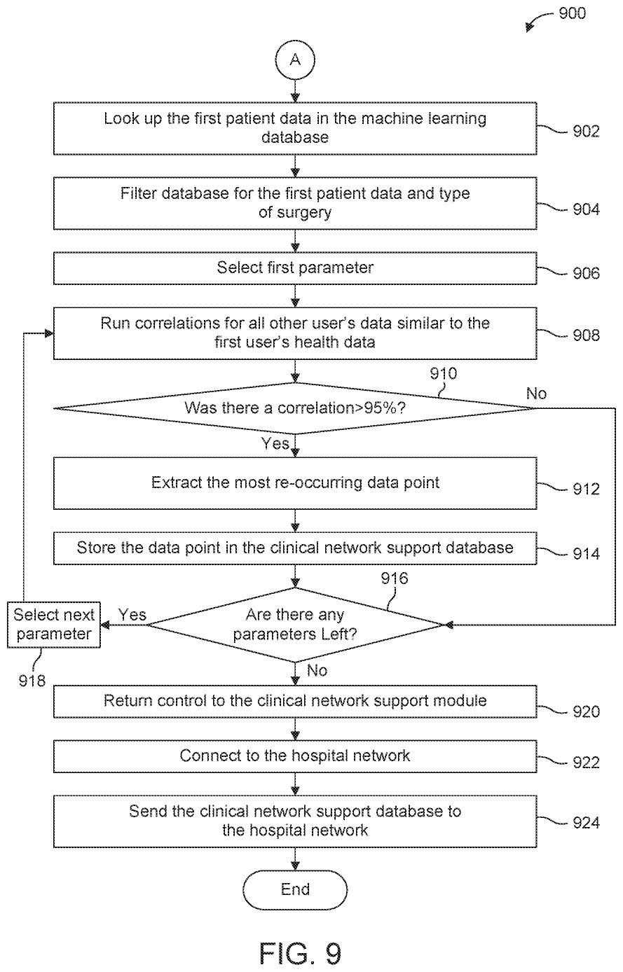

An example process utilizing the clinical machine learning algorithm module 122 will now be explained with reference to, for example, the flowchart 900 shown in FIG. 9. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

Referring to FIG. 9, patient data may be searched in the clinical machine learning database 124 upon receiving the authorized user's health data through the quorum blockchain node, at step 902. The clinical machine learning database 124 may be filtered for the first user's health data and a type of surgery, at step 904. A first parameter of accessible data for the clinical machine learning database 124 may be selected, at step 906. The clinical machine learning database 124 may then be filtered based on the first user's health data (user's age in one case), type of surgery, and first parameter.

Correlations may be performed for some, substantially all, or all other user's health data that has, for example, the same age, type of surgery, and first parameter, at step 908. The correlations may be compared to a predetermined threshold (e.g., >95% or the like) to determine if they are considered relevant for the first user. If the correlation exceeds the predetermined threshold, the most re-occurring data point may be extracted, at step 912. For example, if patients with the same age, surgery and first parameter usually have a high temperature for the first four days after surgery, there may be a chance of occurrence of fever. The correlated data point may be stored in the clinical network support database 126, at step 914. Successive to step 914 or upon determining that no correlation exceeding the predetermined threshold is present, it may be determined if there are any other parameters remaining in the clinical machine learning database 124, at step 916. If there are parameters remaining, a next parameter may be selected at step 918, and correlations may be run for all the parameters left, at step 908. If there are no correlations remaining, the process may return control to the clinical network support module 120, at step 920. Parameters may be any of predetermined parameters that correspond with the physical and or mental wellness of a patient. Parameters may be in the form of numerical data or descriptive data that correlates to information about a patient, such as weight, height, illnesses, prescription drugs take etc. Parameters may be any data that can be expressed, conveyed and collected.

In various embodiments, while function of the clinical machine learning algorithm module 122 gets completed, the process may revert to the clinical network support module 120, at step 922. Data stored in the clinical network support database 126 may be sent to the hospital network, at step 924.

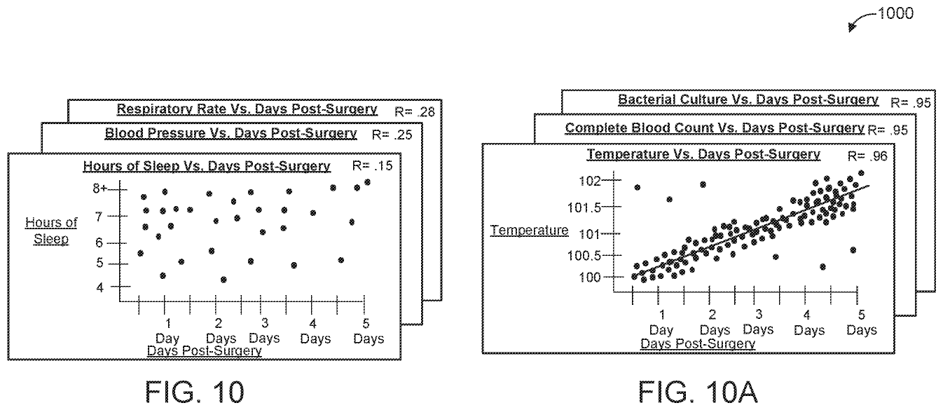

FIGS. 10 and 10A illustrate exemplary data of correlations performed by the clinical machine learning algorithm module 122. Specifically, FIG. 10 illustrates correlations performed between days post-surgery and various parameters such as, for example, hours spent sleeping, blood pressure, and respiratory rate. An example of non-correlated parameters with the days post-surgery may include hours of sleep (an average per night) with a 15% (which is below the 95% threshold). Therefore, no correlation is identified and no data points may be stored in the clinical network support database 126.

FIG. 10A depicts correlations performed between the days post-surgery and various parameters such as, for example, temperature, complete blood count, and bacterial culture. An elevated white blood cell count may be a sign of infection and the bacterial culture is generally performed as a primary test to diagnose a bacterial infection. In various embodiments, the correlations performed between the days post-surgery and temperature had a correlation score of 96%, which is above the 95% threshold. Therefore, a data point corresponding to the correlation may be stored in the clinical network support database 126. The most re-occurring data point, for example, is a temperature of 101.5 lasting for first four days, which may be extracted and stored in the clinical network support database 126. All the correlated data may be viewed by a healthcare practitioner. This may determine that patients with a similar age and same surgery may usually have, for example, fever the first four days after surgery, an elevated white blood cell count, and increase in bacterial culture (e.g., a sign of bacterial infection). The healthcare practitioner may determine from these correlated data points that the patient may have a wound infection and should adjust the process of tending to the wound accordingly.

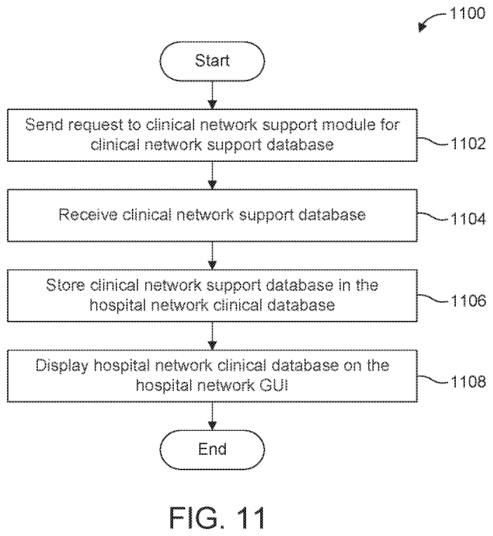

An example process utilizing the Hospital Network Base module 128 will now be explained with reference to, for example, the flowchart 1100 shown in FIG. 11. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

Referring to FIG. 11, a request may be sent to the clinical network support module 120 for exploring data from the clinical network support database 126, at step 1102. The hospital network base module 1100 may then receive the clinical network support database 126, at step 1104. Thereafter, clinical network support database 126 may be stored in the hospital network clinical database 130, at step 1106. The hospital network base module 118 may display the hospital network clinical database to the third party (e.g., healthcare practitioner of the hospital network) for determining if there should be an adjustment made to their workflow to prevent the potential correlated complications, at step 1108.

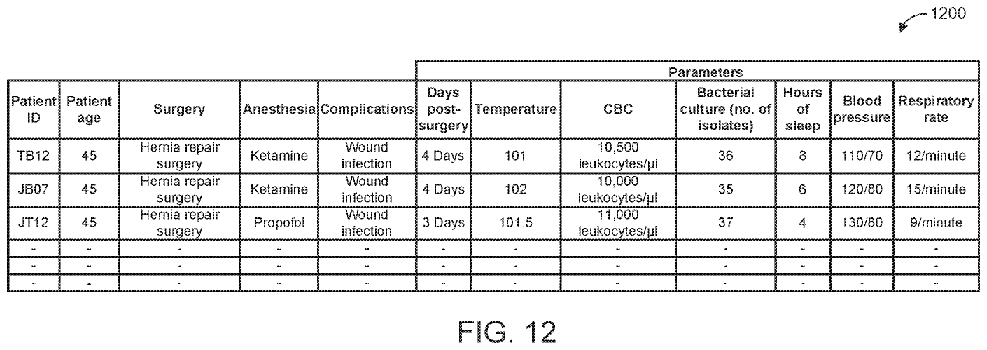

FIG. 12 illustrates exemplary data 1200 stored in the machine learning database 124. The exemplary data 1200 depicts that the user's health data is received from the user's device 104 or another user device using the public key from the Hospital Network. The non-limiting exemplary data 1200 comprises the patient ID, patient age, surgery type, type of anaesthesia, complications, anaesthesia, days post-surgery, patient temperature, Complete Blood Count (CBC), bacterial culture (number of isolates), hours of sleep on average per night, blood pressure, and respiratory rate.

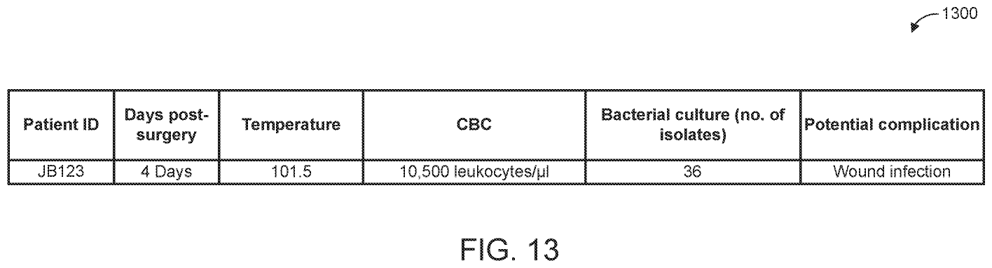

FIG. 13 illustrates exemplary data 1300 stored in the support database 126. In various embodiments, the support database may refer to a clinical network support database. The clinical network support database may store data that may pass a predetermined threshold to determine if there may be a correlation between the data from the clinical machine learning algorithm module 122 by the clinical network support module 120. The clinical network support database may include, for example, patient ID of a concerned patient, the parameter that may be found in a correlation with the pieces of data, other parameters such as temperature, CBC, Bacterial Culture, and the complication that may be associated with these correlated parameters.

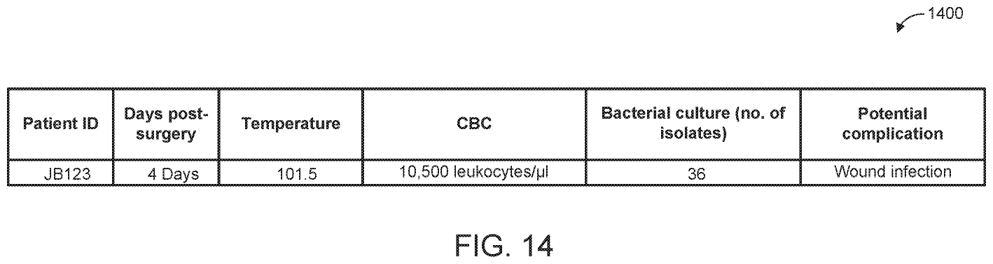

FIG. 14 illustrates exemplary data 1400 stored in a clinical database 130. The clinical database 130 may store the data from the clinical network support module 120. Such data may be displayed to the hospital network for the healthcare practitioner to determine if the correlated data is a potential cause of concern to the user. This may help the healthcare practitioners to adjust their workflow process to prevent occurrence of a disease in the user. The exemplary data 1400 can include, for example, patient ID, a parameter that found a correlation with the pieces of data (for example, days after surgery), other parameters such as temperature, CBC, bacterial culture, and the complication that may be associated with these correlated parameters. Any of the parameters should not be limited to those disclosed herein, as the parameters may be any predetermined data deemed appropriate to collect. Those disclosed were exemplary and parameters may be those beyond what is mentioned.

It will be appreciated that variants of the above disclosed, and other features and functions or alternatives thereof, may be combined into many other different systems or applications. Presently unforeseen or unanticipated alternatives, modifications, variations, or improvements therein may be subsequently made by those skilled in the art that are also intended to be encompassed by the following claims.

What is claimed is:

1. A computer-implemented method of utilizing a user's health data stored over a health care network, for disease prevention, comprising:
allowing a user to manage access of the user's health data to a third party with a user device; and
correlating, using a machine learning algorithm module, the user data with a similar data of other users for suggesting a next best action to be performed by the third party, for preventing occurrence of a disease to the user.

2. The computer-implemented method of claim 1, wherein the similar data of other users is identified based on age, medical conditions, complications after a medical procedure, or treatment.

3. The computer-implemented method of claim 1, wherein the third party comprises at least one of a doctor, a surgeon, and a physician.

4. The computer-implemented method of claim 1, further comprising allowing the third party to look up a first patient data in the machine learning database and to filter the machine learning database for a first patient data and a type of surgery.

5. The computer-implemented method of claim 1, further comprising extracting a most re-occurring data point when a correlation level of the user data with a similar data is greater than a predefined threshold.

6. The computer-implemented method of claim 1, further comprising selecting a new predetermined parameter in the user data for correlating with the similar data when a correlation level of the user data with a similar data is less than a predefined threshold.

7. The computer-implemented method of claim 1, wherein allowing a user to manage access of the user's health data to a third party comprises verifying that the user and the third party have access to a blockchain database storing the user's health data.

8. The computer-implemented method of claim 1, further comprising accessing a digital wallet for the user to update a balance based on a recommendation of the next best action to be performed by the third party.

9. The computer-implemented method of claim 1, wherein correlating the user data with a similar data of other users comprises requesting the similar data from a clinical network support database that runs on a blockchain network.

10. The computer-implemented method of claim 1, wherein suggesting the next best action to be performed by the third party comprises providing a medication schedule to a patient after a surgery based on a likely outcome for other users undergoing a similar surgery.

11. A system for utilizing a user's health data stored over a healthcare network, for disease prevention, comprising:
one or more processors;
a memory coupled to the one or more processors and storing instructions which, when executed by the one or more processors, cause the system to:
allow a user to manage access of the user's health data to a third party with a user device;
correlate, using a machine learning algorithm module, the user data with a similar data of other users for suggesting a next best action to be performed by the third party, for preventing occurrence of a disease to the user; and
allow the third party to look up a first patient data in the machine learning database and to filter the machine learning database for a first patient data and a type of surgery.

12. The system of claim 11, wherein the one or more processors further execute instructions to extract a most re-occurring data point when a correlation level of the user data with a similar data is greater than a predefined threshold.

13. The system of claim 11, wherein the one or more processors further execute instructions to select a new predetermined parameter in the user data for correlating with the similar data when a correlation level of the user data with a similar data is less than a predefined threshold.

14. The system of claim 11, wherein to allow a user to manage access of the user's health data to a third party the one or more processors execute instructions to verify that the user and the third party have access to a blockchain database storing the user's health data.

15. The system of claim 11, wherein the one or more processors further execute instructions to access a digital wallet for the user to update a balance based on a recommendation of the next best action to be performed by the third party.

16. The system of claim 11, wherein to correlate the user data with a similar data of other users the one or more processors further execute instructions to request the similar data from a clinical network support database that runs on a blockchain network.

17. The system of claim 11, wherein to suggest the next best action to be performed by the third party the one or more processors further execute instructions to provide a medication schedule to a patient after a surgery based on a likely outcome for other users undergoing a similar surgery.

18. A non-transitory, computer readable medium storing instructions which, when executed by a processor, cause a computer to perform a method for utilizing a user's health data stored over a healthcare network, for disease prevention, the method comprising:
allowing a user to manage access of the user's health data to a third party with a user device;
correlating and matching, using a machine learning algorithm module, the user data with a similar data of other users for recommending a next best action to be performed by the third party, for preventing occurrence of a disease to the user, said recommendation being ranked based on the matched user data with the matched similar data of others;
allowing the third party to look up a first patient data in the machine learning database and to filter the machine learning database for a first patient data and a type of surgery; and
extracting a most re-occurring data point when a correlation level of the user data with a similar data is greater than a predefined threshold.

19. The non-transitory, computer readable medium of claim 18, further comprising selecting a new predetermined parameter in the user data for correlating with the similar data when a correlation level of the user data with a similar data is less than the predefined threshold.

20. The non-transitory, computer-readable medium of claim 18, wherein allowing a user to manage access of the user's health data to a third party comprises verifying that the user and the third party have access to a blockchain database storing the user's health data.
